(19) United States
(12) Patent Application Publication
(10) Pub. No.: US 2026/0240748 A1
(43) Pub. Date: Aug. 20, 2026

(54) HAIR TREATMENT COMPOSITION

(71) Applicant: Vincent Smith, New York, NY (US)

(72) Inventor: Vincent Smith, New York, NY (US)

(21) Appl. No.: 19/161,037

(22) PCT Filed: Mar. 1, 2024
(86) PCT No.: PCT/US24/18090

§ 371 Date: Aug. 29, 2025

Related U.S. Application Data
(60) Provisional application No. 63/487,869, filed on Mar. 1, 2023.

(51) Int. Cl. A61K 8/73; A61K 8/37; A61K 8/92; A61Q 5/06; A61Q 5/12

(52) U.S. Cl. CPC A61K 8/735; A61K 8/37; A61K 8/922; A61Q 5/06; A61Q 5/12; A61K 2800/882

(57) ABSTRACT

The present invention provides a hair treatment composition including 1) hyaluronic acid, 2) one or more oils comprising triglycerides of C16-C18 fatty acids, which fatty acids may be saturated or unsaturated, 3) preservative, and 4) water. Also provided is a method for treating a keratinous fiber of a subject, the method including applying the hair spray to the keratinous fiber. Also provided is a hair treatment kit including 1) a masque, 2) an oil serum, and 3) a hyaluronic acid serum.

### FIELD OF THE INVENTION

The invention relates to hair treatment compositions and hair treatment kits. The invention also relates to a method for treating the hair of a subject using the hair treatment compositions and kits.

### BACKGROUND OF THE INVENTION

Many different hair care products are used to treat hair. For example, shampoos are used to clean hair, while styling gels are used to preserve hair styling or increase hair manageability. Not all hair care products, however, result in healthy hair or in a user's desired goals. In addition, some hair care products can be expensive to produce, thereby limiting the number of users who can benefit from their routine use.

Hairstyling or treatment products such as hair sprays and conditioners are used for achieving different hair textures, hairstyles and for holding hair strands in place for a period of time. Many commercial hairstyling products include unnecessary ingredients for their functions, and they can sometimes be damaging to hair itself or the scalp. Some of those unnecessary ingredients may pollute air around users or cause an allergic response in some users.

There is a constant need, therefore, for more environmentally friendly, more sustainable, and more user friendly hairstyling or treatment products. However, altering one or more features of a hairstyling or treatment product can be challenging since the interrelationship therebetween affects the product performance. It is therefore important to select a suitable combination and amount of ingredients necessary to achieve the desired performance benefits without negatively affecting the interrelationship therebetween. For hair treatment products, performance benefits may include, for example: excellent hold; long-lasting hold; good humidity resistance; shapeable hold; acceptable drying time; excellent softness, natural hair feel; smooth hair feel; improved hair volume; improved hair shine, acceptable and/or non-stickiness/tackiness of the hands and hair. To consumers, natural hair feel, hair softness, hair volume, hair shine and non-tackiness to the hands and hair is of particular relevance.

### SUMMARY OF THE INVENTION

This disclosure provides hair care compositions having improved properties. The hair care compositions provided herein contain a solution of hyaluronic acid and one or more plant-derived (botanical) oils. In preferred aspects for the compositions and methods provided in this disclosure, each ingredient of the hair spray composition and the hair treatment kit performs an important function with synergistic benefits. Additionally, it is preferred where possible to use ingredients derived from plant-based sources.

The present disclosure provides a hair spray comprising hyaluronic acid and one or more oils in an aqueous composition. The one or more oils may comprise vegetable oils and essential oils. The hair spray composition may comprise hyaluronic acid, one or more vegetable oils, and one or more essential oils in an aqueous composition. The vegetable oils may include argan oil, shea oil, and the like. The essential oil may comprise ylang-ylang oil, orange oil, and the like.

In at least one aspect, the present disclosure provides a hair treatment composition comprising 1) hyaluronic acid, 2) one or more oils comprising triglycerides of C16-C18 fatty acids, which fatty acids may be saturated or unsaturated, 3) optionally, a preservative, and 4) water. In one embodiment, the hair treatment composition is a hair spray. In another embodiment, the hair treatment composition is a lotion. In another embodiment, the hair treatment is a hair masque or may be combined with a hair masque.

Also provided is a method for treating the hair of a subject, the method comprising applying the hair treatment composition to the hair.

Also provided is a hair treatment kit comprising 1) a masque composition, 2) an oil serum, and 3) a hyaluronic acid serum. The masque may comprise 1) one or more C16-C18 fatty alcohols, 2) one or more oils comprising triglycerides of C16-C18 fatty acids, which fatty acids may be saturated or unsaturated, 3) one or more long-chain quaternary ammonium surfactants, 4) dimethicone, 5) isopropyl alcohol, 6) tocopherol, 7) preservative, 8) citric acid, 9) glycerin, and 10) water. The oil serum may comprise one or more oils comprising triglycerides of C16-C18 fatty acids, which fatty acids may be saturated or unsaturated. The hyaluronic acid serum may comprise 1) hyaluronic acid, 2) one or more oils comprising triglycerides of C16-C18 fatty acids, which fatty acids may be saturated or unsaturated, 3) preservative, and 4) water.

### DETAILED DESCRIPTION OF THE INVENTION

Further in relation to this disclosure, before explaining at least the preferred embodiments of the invention in greater detail, it is to be understood that the invention is not limited in its application to the details of construction and to the arrangements of the components set forth in the following description. It would be understood by those of ordinary skill in the art that embodiments beyond those described herein are contemplated, and the embodiments can be practiced and carried out in a plurality of different ways. Also, it is to be understood that the terminology used herein is for the purpose of description and should not be regarded as a limiting factor.

Unless otherwise defined, the terms used herein refer to that which the ordinary artisan would understand such term to mean based on the contextual use of such term herein. To the extent that the meaning of a term used herein as understood by the ordinary artisan based on the contextual use of such term differs in any way from any particular dictionary definition of such term, it is intended that the meaning of the term as understood by the ordinary artisan will prevail.

All percentages are by weight of the total composition/formulation, unless stated otherwise. All ratios are weight ratios, unless stated otherwise. All ranges are inclusive and combinable. The number of significant digits conveys neither a limitation on the indicated amounts nor on the accuracy of the measurements.

All numerical amounts are understood to be modified by the word “about” unless otherwise specifically indicated. As used herein, the term “about” means approximately or nearly and in the context of a numerical value or range set forth herein means 10% of the numerical value or range recited or claimed.

Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Embodiments and aspects described herein may comprise or be combinable with elements or components of other embodiments and/or aspects despite not being expressly exemplified in combination, unless otherwise stated or an incompatibility is stated.

The compositions, methods, uses, and processes herein can comprise, consist of, and consist essentially of the elements and limitations of the invention described herein, as well as any of the additional or optional ingredients, components, steps, or limitations described herein.

As used herein, “consists essentially of” when used in conjunction with a composition means excluding other materials that contribute to the end result of the composition. Small traces that have little or no effect to the treatment as part of the embodiments of the presentation disclosure may exist in a composition that consists essentially of the claimed ingredients under the definition because it would not materially affect its function and/or objective.

The hyaluronic acid compositions, hair spray composition, and hair treatment kit according to the present invention is suitable for application onto any keratinous fiber. In particular, they are stable for use in contact with human hair and the scalp without undue toxicity, incompatibility, instability, allergic response, and the like. Additionally, the compositions provided herein may be suitable for application to the skin, including to the scalp, face, hands, or elsewhere on the body.

The term “substantially free from”, “substantially free of”, or grammatical equivalents thereof, as defined herein means less than about 5%, or less than about 3%, or less than about 1%, or less than about 0.5%, or about 0%.

This disclosure provides methods and materials related to hair care. For example, this document provides hair care compositions and methods for using hair care compositions. The term “hair care composition” as used herein refers to any product that can be used to treat or style hair. Examples of hair care compositions include, without limitation, conditioners, masques, styling gels, styling sprays, styling mousses, styling pomades, leave-in conditioning sprays or lotions, leave-in detangling sprays or lotions, and thermal protection sprays or lotions.

The inventor has surprisingly overcome the above hindrances and answered the aforementioned needs by carefully selecting the specific combination of mutually compatible features such that the interaction therebetween results in a hair treatment compositions and hair treatment kit with good performance.

First, the hair spray composition and hair treatment kit, except the oil serum, pursuant to the present invention is water-based—i.e., it is an aqueous system rather than a predominantly alcohol-based system as is conventionally employed. This water-based system provides safety, sustainability, environmental and cost advantages.

Second, the hair spray composition and hair treatment kit are preferably substantially free of alcohol. This additionally provides consumer-relevant benefits since alcohol (and particularly ethanol) has the reputation of causing the hair to become brittle and dry, particularly for naturally fine and/or dry hair. Without being bound by theory it is believed that low alcohol concentrations help to reduce drying out effect, i.e., reduce a perceived brittle, harsh feel of the hair. Consumers prefer a more natural hair feel and look. The inventor has found that selected hair spray composition and hair treatment kit to achieve, for example, excellent hair feel, particularly natural hair feel, and excellent non-stickiness of the hands and hair, good hold and shapeable hold.

### Hyaluronic Acid

Hyaluronic acid, which may be abbreviated HA, is an anionic, non-sulfated glycosaminoglycan. Hyaluronic acid is a polymer of disaccharides, composed of D-glucuronic acid and N-acetyl-D-glucosamine, linked via alternating β-(1→4) and β-(1→3) glycosidic bonds.

The hyaluronic acid used in the compositions provided herein have a molecular weight between about 2 kilodaltons and about 20 kilodaltons; or between about 2 and about 15 kilodaltons, or between about 2 and about 10 kilodaltons; or between about 8 and about 15 kilodaltons. The hyaluronic acid may have a molecular weight of about 20 kilodaltons or less; or about 15 kilodaltons or less; or about 8 kilodaltons or less; or about 5 kilodaltons or less; or about 3 kilodaltons or less.

The hyaluronic acid may be present in an amount from about 0.5% to about 20% by weight of the composition; or from about 1% to about 10% by weight of the composition; or from about 2% to about 8% by weight of the composition; or from about 4% to about 6% by weight of the composition.

### Oils

The compositions provided herein comprise one or more oils. Preferably the oil component is a natural oil from a botanical source. The oils may comprise one or more vegetable oils and/or essential oils.

The one or more oils may comprise a vegetable oil. The vegetable oil may be a seed oil, i.e., derived from plant seeds, or may be derived from other plant sources such as the fruit. The one or more vegetable oil typically comprises mono-, di- and tri-glycerides of C16-C18 fatty acids, which fatty acids may be saturated or unsaturated. The vegetable oil may be greater than about 90% triglyceride of C16-C18 fatty acids, or may be greater than about 92% triglyceride of C16-C18 fatty acids. The fatty acids may comprise one or more of oleic acid, linoleic acid, palmitic acid, palmitoleic acid, stearic acid, linolenic acid, or mixtures thereof. The one or more vegetable oils may be selected from Argania Spinosa Kernel Oil (Argan Oil), Açaí palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil and mixtures thereof.

The oil component of the composition may also comprise one or more essential oils. The essential oil is a plant-derived natural oil typically obtained by extraction, distillation or pressing and having the characteristic fragrance of the plant or other source from which it is derived. The essential oil may be selected from one or more of Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and mixtures thereof. In embodiments, the essential oil comprises Ylang-Ylang oil, Orange oil or limonene, or combinations thereof.

The one or more essential oil may comprise from about 0.1% to about 20% of the oil component of the composition; or from about 0.1% to about 15% of the oil component of the composition; or from about 0.5 to about 10% of the oil component of the composition. The oil component may also comprise vitamin E oil or D-alpha tocopherol.

### Optional Ingredients

The hair treatment compositions provided herein may contain one or more optional classes of ingredients such pH adjusters, buffers, preservatives, fragrance, and viscosity increasing agents.

### pH Adjustment

The final pH of the hair treatment composition provided herein may be between about 4.0 and about 8.5. To obtain the final pH, the pH of the composition may be optionally adjusted. A pH-adjusting agent may optionally be used to adjust the pH to the desired value. It will be appreciated that the pH adjustment can be accomplished with any of a wide variety of acids or bases. Acids that may be used include citric acid, acetic acid, benzoic acid, glycolic acid, lactic acid, malic acid, and sulfuric acid. Bases that may be used to lower the pH include be potassium hydroxide, potassium carbonate, sodium carbonate, sodium hydroxide, ethanolamine, or triethanolamine.

### Preservatives

To adequately preserve the hair treatment products and prevent microbial growth, a preservative may optionally be included in a hair treatment compositions provided herein. Preservatives may include, benzoic acid, benzyl alcohol, butylparaben, propylparaben, methyparaben, DMDM Hydantoin, potassium benzoate, methyli sothiazolinone, methylchloroisothiazolinone, phenoxyethanol, quaterium-8, quaterium-14, quaterium-15, triclosan, zinc pyrithione, and zinc salicylate.

Each of the features of the hair spray composition and the hair treatment kit, as well as other relevant components, are described in detail hereinafter.

### Hair Spray Composition

The present disclosure provides a hair spray comprising hyaluronic acid and one or more oils in an aqueous composition. The one or more oils may comprise vegetable oils and essential oils.

The hair spray composition may comprise 1) hyaluronic acid, 2) one or more oils comprising triglycerides of C16-C18 fatty acids, which fatty acids may be saturated or unsaturated, 3) optionally, a preservative, and 4) water. Alternatively, the hair spray composition may consist essentially of 1) hyaluronic acid, 2) one or more oils comprising triglycerides of C16-C18 fatty acids, which fatty acids may be saturated or unsaturated, 3) optionally, a preservative, and 4) water. Alternatively, the hair spray composition may consist of 1) hyaluronic acid, 2) one or more oils comprising triglycerides of C16-C18 fatty acids, which fatty acids may be saturated or unsaturated, 3) optionally a preservative, and 4) water.

In embodiments, the hair spray composition comprises 1) hyaluronic acid, 2) one or more oils selected from vegetable oils, essential oils, or combinations thereof, 3) optionally, a preservative, and 4) water.

The hyaluronic acid may have a molecular weight of about 8 kilodaltons or less, or about 5 kilodaltons or less. The inventor discovered that hyaluronic acid having a lower molecular weight, for example about 5 kilodaltons is more suitable for a spray or other liquid hair treatment. On the other hand, a molecular weight of about 8 to about 20 kilodaltons may be more suitable for a gel treatment.

The hyaluronic acid may be present in an amount from about 0.1% to about 20% by weight of the composition. Preferably, the hyaluronic acid may be present in an amount from about 1% to about 10% by weight of the composition; or from about 2% to about 8% by weight of the composition; or from about 4% to about 6% by weight of the composition.

The one or more oils may include triglycerides of oleic acid, linoleic acid, palmitic acid, palmitoleic acid, stearic acid, linolenic acid, or mixtures thereof. The one or more oils may be derived from a botanical source.

The one or more oils may comprise one or more vegetable oils, one or more essential oils, and combinations thereof. The one or more oils may be present in an amount from about 0.1% to about 10% by weight of the composition. Preferably, the one or more oils may be present in an amount from about 1% to about 5% by weight of the composition; or from about 1% to about 4% by weight of the composition.

The one or more vegetable oils may include triglycerides of oleic acid, linoleic acid, palmitic acid, palmitoleic acid, stearic acid, linolenic acid, or mixtures thereof, and may be selected from Argania Spinosa Kernel Oil (Argan Oil), Açaí palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil and mixtures thereof. Preferred vegetable oils include one or more of argan oil, shea oil, and avocado oil. The vegetable oil may be present in the spray composition in an amount from about 0.1% to about 10% by weight of the composition. Preferably, the one or more oils may be present in an amount from about 1% to about 5% by weight of the composition; or from about 1% to about 4% by weight of the composition.

The hair spray composition may also comprise one or more essential oils. The essential oil may be selected from, but are not limited to, one or more of Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and mixtures thereof. Preferred essential oils may include Orange oil, limonene, Ylang-ylang oil, and combinations thereof. The essential oil may be present in the hair spray composition in an amount from about 0.1% to about 5% by weight of the composition; or the one or more essential oils may be present in an amount from about 0.2% to about 4% by weight of the hair spray composition; or the one or more essential oils may be present in an amount from about 0.2% to about 3% by weight of the hair spray composition; or the one or more essential oils may be present in an amount from about 1% to about 3% by weight of the hair spray composition.

The hair spray composition is sprayed onto the subject's hair, which may then be styled as desired by the subject. The hair spray may be applied to wet hair, for example to damp or towel-dried hair. In aspects, the hair spray is applied to the subject's hair, which may then be styled by the application of heat to the hair, for example, by use of a flat iron or a blow dryer.

### Hair Spray Compositions:

| Ingredients | % by weight |
|---|---|
| Preservative(s) | 0-1.0% |
| Hyaluronic Powder | 1-10% |
| Vegetable Oil(s) | 0.1-10% |
| Essential Oil(s) | 0.1-5% |
| Water | up to 100% |

| Ingredients | % by weight |
|---|---|
| Preservative(s) | 0.1-1.0% |
| Hyaluronic Powder | 2-8% |
| Vegetable oil(s) | 1-5% |
| Essential Oil(s) | 0.2-4% |
| Water | up to 100% |

| Ingredients | % by weight |
|---|---|
| Preservative | 0.1-1.0% |
| Hyaluronic Powder | 2-8% |
| Argan Oil | 1-5% |
| Orange Oil | 0.5-3% |
| Ylang Ylang Oil | 0.2-1% |
| Water | up to 100% |

Compared to conventional hair spray compositions, this hair spray composition according to the present disclosure showed surprising performance in terms of imparting additional volume and softness without the usual stickiness or stiffness imparted with other volumizing sprays. On naturally curly and wavy hair, it enhances the curl, wave and volume, while imparting additional volume. This benefit is achieved because HA has the unique ability to attract and retain many times it's weight in water, thereby plumping up the hair shaft and enhancing volume and curl, where present.

The spray composition may also be applied to the subject's skin, including the hands, face and other areas to promote excellent moisturization of the skin and skin softness.

### Hair Treatment Kit

Also provided is a hair treatment kit comprising three components: 1) a masque, 2) an oil serum, and 3) a hyaluronic acid serum. The hair treatment kit may further comprise an item such as, but not limited to, a shampoo, conditioner, mousse, gel, a hairstyling tool, blow dryer, curling tongs, and straightening irons. The hair styling tool may include hair bands, hair fasteners, combs, and brushes.

### Masque

The hair masque component comprises a deep conditioning treatment for the hair. The masque may increase moisture in hair, prevent breakage and help prevent frizz, and also may make hair soft and shiny. The masque component of the kit may be any masque composition known in the art. Preferably, the ingredients of the masque composition are derived, where possible, from plant-based sources.

The masque component may comprise one or more of: 1) one or more C16-C18 fatty alcohols, 2) one or more oils comprising triglycerides of C16-C18 fatty acids, which fatty acids may be saturated or unsaturated, 3) one or more long-chain quaternary ammonium surfactants, 4) dimethicone, 5) isopropyl alcohol, 6) tocopherol, 7) preservative, 8) citric acid, 9) glycerin (or glycerol), and 10) water.

The one or more C16-C18 fatty alcohols of the masque may include cetearyl alcohol and cetyl alcohol.

For cetearyl alcohol, a mixture of fatty alcohols, consisting predominantly of cetyl (C16) and stearyl alcohols (C18) may be used. It may be used as an emulsion stabilizer, opacifying agent, and foam boosting surfactant, as well as an aqueous and nonaqueous viscosity-increasing agent.

The one or more C16-C18 fatty alcohols of the masque may be present in an amount from about 1% to about 10% by weight of the masque.

The masque comprises one or more vegetable oils. The one or more vegetable oils may be selected from Argania Spinosa Kernel Oil (Argan Oil), Açai palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil and mixtures thereof. The masque may also comprise one or more essential oils. The essential oil may be selected from one or more of Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and mixtures thereof.

The one or more oils may comprise triglyceride with α-linolenic acid (e.g., about 51.9-55.2%), palmitic acid (e.g., about 7%), stearic acid (e.g., about 3.4-4.6%), monounsaturated oleic acid (e.g., about 18.5-22.6%), and doubly unsaturated linoleic acid (e.g., about 14.2-17%).

For example, Argan Oil may comprise oleic acid (18:1 cis-9) (e.g., about 42.8%), linoleic (18:2 n-6) (e.g., about 36.8%), palmitic acid (16:0) (e.g., about 12.0%), stearic acid (18:0) (e.g., about 6.0%), and linolenic acid <about 0.5%.

For example, orange oil may be composed of mostly (e.g., greater than 90%) d-limonene. It functions as an essential oil, scent, and solvent. Similarly, Ylang Ylang Oil functions as an essential oil and scent.

The one or more oils of the masque may be present in an amount from about 0.1% to about 15% by weight of the masque, or from about 1% to about 10%, or from about 3% to about 8%. Alternatively, the one or more oils of the masque may be present in an amount from about 1% to about 5% by weight of the masque. Alternatively, the one or more oils of the masque may be present in an amount from about 5% to about 15% by weight of the masque.

The one or more long-chain quaternary ammonium surfactants of the masque may include cetrimonium chloride (or cetyltrimethylammonium chloride (CTAC)) and behentrimonium chloride (or docosyltrimethylammonium chloride or BTAC-22). Cetrimonium chloride may be a topical antiseptic and surfactant. Long-chain quaternary ammonium surfactants, such as cetyltrimethylammonium chloride (CTAC), may be generally combined with long-chain fatty alcohols, such as stearyl alcohols, in formulations of hair conditioners and shampoos.

The long-chain quaternary ammonium surfactants of the masque may be present in an amount from about 0.1% to about 10% by weight of the masque, or from about 1% to about 10%. Preferably, the long-chain quaternary ammonium surfactants of the masque may be present in an amount from about 1% to about 5% by weight of the masque.

Dimethicone, also known as polydimethylsiloxane (PDMS) and dimethylpolysiloxane may belong to a group of polymeric organosilicon compounds that are commonly referred to as silicones. It may be one of several types of silicone oil (polymerized siloxane). It may be used to add shine to the keratinous fiber.

The preservative of the masque may include DMDM Hydantoin. DMDM Hydantoin, known as trade name Glydant, may function as a preservative or an antimicrobial formaldehyde releaser. The preservative of the masque may be present in an amount from about 0.1% to about 5% by weight of the masque. Preferably, the preservative of the masque may be present in an amount up to about 3% by weight of the masque.

The water of the masque may include distilled or purified water. The water of the masque may be present in an amount not less than about 60% to about 80% by weight of the masque.

Additional ingredients such as, but not limited to, Moihm02, alpha-isomethyl ionone, linalool, or butylphenyl methylproponal may be added to the masque for scent or other desired purposes.

### Masque of Hair Treatment Kit

| Ingredients | % by weight | % by weight | % by weight |
|---|---|---|---|
| glycerin | 0-5% | 1-3% | 1-3% |
| C16-C18 fatty alcohols | 1-10% | 2-6% | 2-6% |
| vegetable oils | 1-10% | 3-8% | 3-8% |
| essential oils | 0-6% | 0.1-1% | 1-6% |
| dimethicone | 0-5% | 1-4% | 1-4% |
| long-chain quaternary | 0.1-10% | 2-5% | 2-5% |
| ammonium surfactants | | | |
| isopropyl or ethyl alcohol | 0-3% | 0-1% | 0.5-2% |
| D-alpha tocopherol | 0-2% | 0.5-1.5% | 0-1.5% |
| preservative | 0-1% | 0.1-0.75% | 0.1-0.75% |
| acid for pH adjustment | 0-4% | 0-0.75% | 0-3% |
| water | to 100% | to 100% | to 100% |

### Oil Serum

The second component of the kit, the oil serum, may include one or more oils comprising triglycerides of C16-C18 fatty acids, which fatty acids may be saturated or unsaturated. The oils are preferably derived from a botanical source.

The one or more oils may comprise one or more vegetable oils, one or more essential oils, and combinations thereof. The one or more oils may be present in an amount from about 50% to about 100% by weight of the oil serum composition. Preferably, the one or more oils may be present in an amount from about 70% to about 100% by weight of the oil serum composition; or the one or more oils may be present in an amount from about 90% to about 100% by weight of the oil serum composition; or the one or more oils may be present in an amount from about 95% to about 100% by weight of the oil serum composition.

The one or more vegetable oils may include triglycerides of oleic acid, linoleic acid, palmitic acid, palmitoleic acid, stearic acid, linolenic acid, or mixtures thereof, and may be selected from Argania Spinosa Kernel Oil (Argan Oil), Açaí palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil and mixtures thereof. Preferred vegetable oils include argan oil, shea oil and avocado oil. The vegetable oil may be present in the serum composition in an amount from about 50% to about 100% by weight of the composition. Preferably, the one or more vegetable oils may be present in an amount from about 60% to about 90% by weight of the composition; or the one or more vegetable oils may be present in an amount from about 70% to about 90% by weight of the composition; or the one or more vegetable oils may be present in an amount from about 70% to about 95% by weight of the composition.

The oil serum composition may also comprise one or more essential oils. The essential oil may be selected from, but are not limited to, one or more of Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and combinations thereof. The essential oil may be present in the oil serum in an amount from about 0% to about 30% by weight of the composition; or the one or more essential oils may be present in an amount from about 2% to about 30% by weight of the oil serum composition; or the one or more essential oils may be present in an amount from about 5% to about 25% by weight of the oil serum composition; or the one or more essential oils may be present in an amount from about 10% to about 30% by weight of the oil serum composition.

The oil serum may optionally comprise vitamin E oil or D-alpha tocopherol. The vitamin E or D-alpha tocopherol may be present in an amount from about 0% to about 20% by weight of the oil serum composition; or from about 5% to about 15% by weight of the oil serum composition; or from about 0% to about 15% by weight of the oil serum composition.

### Oil Serum of Hair Treatment Kit

| Ingredients | % by weight | % by weight | % by weight |
|---|---|---|---|
| Vegetable oils | 50-100% | 60-90% | 70-90% |
| Essential oils | 0-30% | 5-25% | 10-30% |
| Vitamin E Oil | 0-20% | 5-15% | 0-15% |

Compared to conventional oil serum, this oil serum according to the present disclosure showed surprising performance in terms of defrizzing, smoothing, softening enhanced ease of styling and hold because of the ease of absorption and aid in closing the cuticle of the hair shaft.

Additionally, the oil serum composition may also be applied to the subject's skin, including the hands, face and other areas to promote excellent moisturization of the skin and skin softness.

### Hyaluronic Acid Serum

The third component of the kit, the hyaluronic acid serum, comprises a water-based hyaluronic acid solution. The hyaluronic acid may have a molecular weight between about 2 kilodaltons and about 20 kilodaltons; or between about 2 and about 15 kilodaltons, or between about 2 and about 10 kilodaltons; or between about 8 and about 15 kilodaltons. The hyaluronic acid may have a molecular weight of about 20 kilodaltons or less; or about 15 kilodaltons or less; or about 8 kilodaltons or less; or about 5 kilodaltons or less; or about 3 kilodaltons.

The hyaluronic acid of the hyaluronic acid serum may be present in an amount from about 0.1% to about 25% by weight of the hyaluronic acid serum; or from about 1% to about 20%. Preferably, the hyaluronic acid of the hyaluronic acid serum may be present in an amount from about 5% to about 15% by weight of the hyaluronic acid serum; or from about 3% to about 10% by weight of the hyaluronic acid serum.

The hyaluronic acid serum may optionally comprise one or more oils. The one or more oils of the hyaluronic acid serum are selected from Argania Spinosa Kernel Oil (Argan Oil), Ylang Ylang Oil, Flaxseed Oil (Linseed Oil), Orange Oil, Shea Nut Oil, Olive Oil, Avocado Oil, Apricot Seed Oil, Jojoba Oil, Shea Oil, Coconut Oil, Almond Oil, African Galanga Oil, Ama Oil, Asian Borage Oil, Camellia Oil, Orchid Oil, Castor Oil, Marula Oil, Grapeseed Oil, Tea Tree Oil, Vitamin E Oil, and combinations thereof. Preferably, the one or more oils of the hyaluronic acid serum comprises Argan Oil, Shea Nut Oil, Vitamin E oil, Avocado Oil, Orange Oil and Ylang Ylang Oil. The one or more oils of the hyaluronic acid serum may comprise Argan Oil Orange oil, Ylang Ylang Oil, and combinations thereof.

The one or more oils of the hyaluronic acid serum may be present in an amount from about 0% to about 10% by weight of the hyaluronic acid serum. Preferably, the one or more oils of the hyaluronic acid serum may be present in an amount from about 0% to about 5% by weight of the hyaluronic acid serum.

The preservative of the hyaluronic acid serum may include DMDM hydantoin, imadozolidinyl urea, diazolidinyl urea, phenoxy ethanol, caprylyl glycol, methylisothiazolinone, benzoic acid, sorbic acid, levulinic acid, anisic acid, methylparaben, ethylparaben, propylparaben, butylparaben, isobutylparaben, benzyl alcohol, piroctone olamine, or combinations thereof.

The preservative of the hyaluronic acid serum may include benzyl alcohol. The preservative of the hyaluronic acid serum may be present in an amount from about 0.1% to about 5% by weight of the hyaluronic acid serum. The preservative of the hyaluronic acid serum may be present in an amount up to about 3% by weight, or up to about 2% by weight, of the hyaluronic acid serum.

The water of the hyaluronic acid serum may include distilled water. The water may be present in an amount not less than about 60% by weight of the hyaluronic acid serum. The water may be present in an amount not less than about 80% by weight of the hyaluronic acid serum.

### Hyaluronic Acid Serum of Hair Treatment Kit

| Ingredients | % by weight | % by weight | % by weight |
|---|---|---|---|
| hyaluronic powder | 1-20% | 5-15% | 3-10% |
| preservative | 0.1-5% | 0-2% | 0.1-1% |
| oil | 0-10% | 0-5% | 0-5% |
| water | to 100% | to 100% | to 100% |

The masque, oil serum and hyaluronic acid serum are combined prior to the application to the hair. The relative amounts of the masque, oil and hyaluronic acid may be adjusted to suit the hair of the subject. In embodiments, about 84 to about 112 grams masque, about 100 to 300 ml oil serum, and about 100 ml of hyaluronic acid serum are mixed and applied to the hair. For more volume, the hyaluronic acid serum may be increased to about 400 ml. For hair that tends to frizz and expand in a humid atmosphere, the hyaluronic acid serum may be omitted from the mixture.

The combined masque, oil, and hyaluronic acid may be applied to wet or dry hair. The treatment may work best when applied to clean, towel-dried hair that is still damp.

Preferably, after the combination is applied to the hair, the method further includes a step in which stream is applied to the hair. After the mixture is applied to the hair, it may be steamed into the hair, for example with a flat iron that exudes steam when pressed into the hair. This allows the treatment to penetrate the hair shaft thereby creating lasting results of detangling, incredible softness, shine, increased volume and enhanced curl and wave. Following the steam treatment, the hair treatment is rinsed from the hair, after which the hair is styled as desired. The results of the process may last up to six months or more.

### Methods for Treating a Keratinous Fiber of a Subject

Also provided is a method for treating a keratinous fiber or a scalp of a subject. The method may include applying the hair spray composition and/or a hair treatment kit described herein. It may be contemplated that both the hair spray composition and the hair treatment kit can be used simultaneously or in sequence in any order.

The method may further include a step of washing or rinsing the keratinous fiber or the scalp prior to applying the hair spray composition or the hair treatment kit. Liquid shampoo, Solid shampoo, soap, foaming hair cleaner, rinse, conditioner, or a similar item may be used for this step. This step may help clean the keratinous fiber or scalp by removing oils and debris, thereby maximizing the benefits of the composition or the hair treatment kit. The washing or rinsing step may be done according to any known or to be known fashion in various approaches.

The method may further include a step of applying stream to the hair. After a hair treatment composition as provided herein is applied to the hair, it may be steamed into the hair, for example with a flat iron that exudes steam when pressed into the hair. This allows the treatment to penetrate the hair shaft thereby creating lasting results of detangling, incredible softness, shine, increased volume and enhanced curl and wave. The results of the process may last up to six months or more.

The method may further include a step of styling the keratinous fiber or the scalp. The hairstyling can be done by a user of the hair treatment kit or by a stylist. The hairstyling step may be done according to any known or to be known fashion in various approaches.

When using the hair treatment kit, although it is possible to use all of the components, for example, masque, oil serum, and hyaluronic acid serum, it is not necessary to use all of them. A user can choose which one to use to suit his or her requirements.

Any additional step or steps to complete the hair treatment may be added to the method described herein. Any steps described herein may be performed in any order, with or without duplication ore repetition.

The advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims

### EXAMPLES

### Example 1—Hyaluronic Acid Hair Spray

To make the HA spray, the Benzyl Alcohol is mixed with the water. Then the HA powder is sprinkled on top of the water and not stirred. The mixture is then covered in an airtight container and refrigerated for 2 to 4 hours until the water is absorbed by the HA, which is then dissolved into solution. Then the mixture is stirred, and the oils are added. The mixture is stirred again and put into a spray bottle. No propellants or fluorocarbons are added, maintaining the purity of the product.

### Hair Spray Composition

| | Ingredients | % by weight |
|---|---|---|
| Phase A | Distilled Water | 90.0% |
| Phase A | Benzyl Alcohol | 0.500% |
| Phase B | Hyaluronic Powder | 5.00% |
| Phase C | Argan Oil | 2.00% |
| Phase C | Orange Oil | 2.00 |
| Phase C | Ylang Ylang Oil | 0.500% |
| | Total | 100% |

### Example 2—Hyaluronic Acid Hair Spray

The HA Spray composition may be prepared according to the method provided in Example 1.

### Hair Spray Composition

| | Ingredients | % by weight |
|---|---|---|
| Phase A | Distilled Water | 91.0% |
| Phase A | Benzyl Alcohol | 0.500% |
| Phase B | Hyaluronic Powder | 5.00% |
| Phase C | Argan Oil | 1.60% |
| Phase C | Orange Oil | 1.50 |
| Phase C | Ylang Ylang Oil | 0.400% |
| | Total | 100% |

The HA Spray of Examples 1 and 2 are generously sprayed into clean, damp (i.e., towel dried) hair and worked through by hand, scrunched in, combed or brushed through. Style as normal or allow to dry naturally. The HA spray can be used as a volumizing spray for anyone that wants more volume. It leaves the hair soft and weightless with no tackiness, stickiness or crunchiness.

The HA spray imparts volume, softness and shine. On naturally curly and wavy hair, the HA spray enhances the natural curl and imparts volume and softness. It leaves the hair extremely soft without stickiness or stiffness like other volume sprays. It can also be used as a curl enhancing spray. The hydration provided by the Hyaluronic Acid Serum supports and defines curls while adding body. The Aran Oil in the formula further hydrates and prevents frizz.

### Example 3—Hair Treatment Kit

The masque is prepared by emulsifying the Behentrimonium Chloride, the Cetyl and Cetearyl Alcohols and the Citric Acid over heat. When blended, the Glycerin, Dimethicone, and Cetrimonium Chloride are added and blended. The alcohol and preservative (i.e., Benzyl Alcohol, Germall Plus or Optiphen Plus) are added to the water, then that mixture is blended into the emulsified mixture. Then the Argania Spinosa Oil, Flax Seed Oil, D-Alpha Tocopherol (Vitamin E), Orange Oil and Ylang Ylang are added and blended into the mixture, which is then allowed to cool before packaging and use.

### Masque Composition

| | Ingredients | % by weight |
|---|---|---|
| Phase A | Water | 80.0% |
| Phase A | Glycerin | 2.00% |
| Phase B | Cetearyl Alcohol | 3.00% |
| Phase B | Argania Spinosa Kernel Oil | 4.00% |
| Phase B | Dimethicone | 2.00% |
| Phase B | Cetrimonium Chloride Cetac-30 | 2.00% |
| Phase B | Cetyl Alcohol | 1.00% |
| Phase B | Behentrimonium Chloride | 2.00% |
| Phase B | Isopropyl Alcohol | 0.50% |
| Phase B | Flax Seed Oil | 1.00% |
| Phase B | D-Alpha Tocopherol | 1.00% |
| Phase C | Ylang Ylang Oil | 0.100% |
| Phase C | Orange Oil | 0.400% |
| Phase D | Preservative | 0.500% |
| Phase D | Citric Acid | 0.500% |
| | Total | 100% |

### Hyaluronic Acid Serum

| | Ingredients | % by weight |
|---|---|---|
| Phase A | Distilled Water | 94.5% |
| Phase A | Benzyl Alcohol | 1.00% |
| Phase B | Pure Hyaluronic Powder | 10.0% |
| | Total | 100% |

The Oil Serum is prepared by adding all the ingredients together and agitating to mix.

### Oil Serum of Hair Treatment Kit

| | Ingredients | % by weight |
|---|---|---|
| Phase A | Argan Oil | 44.8% |
| Phase A | Shea Nut Oil | 33.6% |
| Phase B | Vitamin E Oil | 11.2% |
| Phase B | Avocado Oil | 5.60% |
| Phase B | Orange Oil | 11.2% |
| Phase B | Ylang Ylang Oil | 5.60% |
| | Total | 100% |

### Example 4—Hair Treatment Kit

The hair treatment may be prepared according to the procedure provided in Example 3.

### Masque Composition

| | |
|---|---|
| % | Ingredients |

PHASE A

| | |
|---|---|
| 2.821% | Behentrimonium Chloride |
| 2.821% | Cetearyl Alcohol |
| 2.821% | Cetyl Alcohol |
| 2.821% | Shea Butter |

PHASE B

| | |
|---|---|
| 1.410% | Glycerin |
| 1.410% | Centrimonium Chloride |
| 1.410% | Dimethicone |

PHASE C

| | |
|---|---|
| 14.104% | Water |
| 1.410% | White Vinegar |
| 1.410% | Ethyl Alcohol |
| 1.410% | Benzyl Alcohol |
| 1.410% | Citric Acid |

PHASE D

| | |
|---|---|
| 1.410% | Argan Oil |
| 0.564% | Avocado Oil |
| 0.423% | Flax Seed Oil |
| 0.564% | Ylang Ylang Oil |
| 1.410% | Orange Oil |

PHASE E

| | |
|---|---|
| 56.417% | Distilled Water |

PHASE F

| | |
|---|---|
| 1.410% | Argan Oil |
| 1.410% | Orange Oil |
| 0.282% | Ylang Ylang |
| 0.141% | Cedarwood |
| 0.141% | Vanilla |
| 0.141% | Vetiver |
| 0.141% | Frankinsence |
| 0.282% | Myrrh |
| 100% | Total |

### Hyaluronic Acid Serum

| Ingredients | % by weight |
|---|---|
| Distilled Water | 94.5% |
| Benzyl Alcohol | 0.50% |
| Pure Hyaluronic Powder | 5.0% |
| Total | 100% |

The Oil Serum used in the preparation may be Composition A or B:

### Oil Serum Composition A

| % by wt. | Ingredients |
|---|---|
| | Phase A |
| 45.000% | Argan Oil |
| 30.000% | Shea Nut Oil |
| | Phase B |
| 10.000% | Avocado Oil |
| 10.000% | Orange Oil |
| 5.000% | Ylang Ylang Oil |

### Oil Serum Composition B

| % By Wt. | Ingredients |
|---|---|
| 38.095% | Argan Oil |
| 28.571% | Shea Nut Oil |
| 9.524% | Avocado Oil |
| 19.048% | Orange Oil |
| 4.762% | Ylang Ylang Oil |

The masque, oil serum and hyaluronic acid serum compositions of Example 3 or 4 are combined prior to the application to the hair. Preferably, mix the oil serum and hyaluronic acid serum into the treatment masque. The relative amounts of the masque, oil and hyaluronic acid may be adjusted to suit the hair of the subject. About 84 to about 112 grams masque, about 100 to 300 ml oil serum, and about 100 ml of hyaluronic acid serum are mixed and applied to the hair. For more volume, the hyaluronic acid serum may be increased to about 400 ml. For hair that tends to frizz and expand in a humid atmosphere, the hyaluronic acid serum may be reduced or omitted from the mixture.

The combined masque, oil, and hyaluronic acid may be applied to clean, towel-dried hair that is still damp, and combed through with a fine-tooth comb for even distribution.

After the mixture is applied to the hair, it is steamed into the hair using a flat iron that exudes steam when pressed into the hair. This allows the treatment to penetrate the hair shaft thereby creating lasting results of detangling, incredible softness, shine, increased volume and enhanced curl and wave. Following the steam treatment, the hair treatment is rinsed from the hair, after which the hair is styled as desired, for example by allowed the hair to dry naturally or blow drying to the desired style.

The results that the client or at-home user experiences after administering the treatment kit is extreme hydration of the hair without any added weight, lasting detangling up to 6 months to a year. The hair is softened to the equivalent of the feel of the finest cashmere. Shine is imparted. Natural curl and wave are enhanced. Fine and limp hair experience more volume and fullness. Hair with damaged and abraded ends experience sealing of the ends.

Each of the oil serums of Examples 3 and 4 may be used alone on the hair or skin. For example, on damp hair, put 2 to 4 drops into the palm of your hand. Emulsify and distribute throughout the hair, preferably avoiding the roots. Style as normal. The oil serums will soften and hydrate the hair and tame frizz. They can be used sparingly on dry hair to further tame frizz.

What is claimed is:

1. A hair treatment composition comprising:
hyaluronic acid,
one or more oils comprising triglycerides of C16-C18 fatty acids, which fatty acids may be saturated or unsaturated,
optionally, a preservative, and
water.

2. The hair treatment composition of claim 1, wherein the hyaluronic acid has a molecular weight of about 20 kilodaltons or less; or about 15 kilodaltons or less; or about 8 kilodaltons or less; or about 5 kilodaltons or less; or about 3 kilodaltons or less.

3. The hair treatment composition of claim 1, wherein the hyaluronic acid has a molecular weight of about 5 kilodaltons or less; or about 3 kilodaltons or less.

4. The hair treatment composition according to any one of claims 1 to 3, wherein the hyaluronic acid is present in an amount from about 0.5% to about 20% by weight of the composition; or from about 1% to about 10% by weight of the composition; or from about 2% to about 8% by weight of the composition; or from about 4% to about 6% by weight of the composition.

5. The hair treatment composition according to any one of claims 1 to 4, wherein the hyaluronic acid is present in an amount from about 1% to about 10% by weight of the composition; or from about 2% to about 8% by weight of the composition; or from about 4% to about 6% by weight of the composition.

6. The hair treatment composition according to any one of claims 1 to 5, wherein the one or more oils comprise triglycerides of oleic acid, linoleic acid, palmitic acid, palmitoleic acid, stearic acid, linolenic acid, or mixtures thereof.

7. The hair treatment composition according to any one of claims 1 to 6, wherein the one or more oils comprise vegetable oils selected from Argania Spinosa Kernel Oil (Argan Oil), Açaí palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil and mixtures thereof.

8. The hair treatment composition according to any one of claims 1 to 7, wherein the one or more oils comprise essential oils selected from Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and mixtures thereof.

9. The hair treatment composition according to any one of claims 1 to 8, wherein the one or more oils comprise one or more of Argan Oil, Orange Oil and Ylang Ylang Oil.

10. The hair treatment composition according to any one of claims 1 to 9, wherein the one or more oils are present in an amount from about 0.1% to about 10% by weight of the composition; or the one or more oils are present in an amount from about 1% to about 5% by weight of the composition.

11. The hair treatment composition according to any one of claims 1 to 9, wherein the vegetable oil is present in the composition in an amount from about 0.1% to about 10% by weight of the composition; or in an amount from about 1% to about 5% by weight of the composition; or from about 1% to about 4% by weight of the composition.

12. The hair treatment composition according to any one of claims 1 to 11, wherein one or more essential oils are present in the composition in an amount from about 0.1% to about 5% by weight of the composition; or an amount from about 0.2% to about 4% by weight of the composition; or in an amount from about 0.2% to about 3% by weight of the composition; or in an amount from about 1% to about 3% by weight of the composition.

13. The hair treatment composition according to any one of claims 1 to 12, wherein the preservative comprises an aldehyde, a glycol ether, an isothiazolinone, an organic acid, a paraben, and combinations thereof.

14. The hair treatment composition according to any one of claims 1 to 13, wherein the preservative comprises DMDM hydantoin, imadozolidinyl urea, diazolidinyl urea, phenoxy ethanol, caprylyl glycol, methylisothiazolinone, benzoic acid, sorbic acid, levulinic acid, anisic acid, methylparaben, ethylparaben, propylparaben, butylparaben, isobutylparaben, benzyl alcohol, piroctone olamine, or combinations thereof.

15. The hair treatment composition of claim 14, wherein the preservative comprises benzyl alcohol.

16. The hair treatment composition according to any one of claims 1 to 15, wherein the preservative is present in an amount from about 0.1% to about 5% by weight of the composition.

17. The hair treatment composition according to any one of claims 1 to 15, wherein the hair treatment is a hair spray.

18. The hair treatment composition according to any one of claims 1 to 17, consisting essentially of: hyaluronic acid; one or more oils comprising triglycerides of C16-C18 fatty acids, which fatty acids may be saturated or unsaturated; optionally a preservative; and water.

19. The hair treatment composition according to any one of claims 1 to 17, consisting of: hyaluronic acid; one or more oils comprising triglycerides of C16-C18 fatty acids, which fatty acids may be saturated or unsaturated; optionally a preservative; and water.

20. A hair treatment composition comprising:
hyaluronic acid,
one or more oils comprising a vegetable oil and an essential oil;
optionally, a preservative, and
water.

21. The hair treatment composition of claim 20, wherein the hyaluronic acid has a molecular weight of about 20 kilodaltons or less; or about 15 kilodaltons or less; or about 8 kilodaltons or less; or about 5 kilodaltons or less; or about 3 kilodaltons or less.

22. The hair treatment composition of claim 20, wherein the hyaluronic acid has a molecular weight of about 8 to about 15 kilodaltons.

23. The hair treatment composition according to any one of claims 20 to 22, wherein the hyaluronic acid is present in an amount from about 0.5% to about 20% by weight of the composition; or from about 1% to about 10% by weight of the composition; or from about 2% to about 8% by weight of the composition; or from about 4% to about 6% by weight of the composition.

24. The hair treatment composition according to any one of claims 20 to 23, wherein the hyaluronic acid is present in an amount from about 1% to about 10% by weight of the composition.

25. The hair treatment composition according to any one of claims 20 to 24, wherein the one or more vegetable oils comprise triglycerides of oleic acid, linoleic acid, palmitic acid, palmitoleic acid, stearic acid, linolenic acid, or mixtures thereof.

26. The hair treatment composition according to any one of claims 20 to 25, wherein the one or more vegetable oils is selected from Argania Spinosa Kernel Oil (Argan Oil), Açaí palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil and mixtures thereof.

27. The hair treatment composition claim 26, wherein the vegetable oil comprises argan oil and/or shea oil.

28. The hair treatment composition according to any one of claims 20 to 27, wherein the one or more essential oil is selected from Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and mixtures thereof.

29. The hair treatment composition according to any one of claims 20 to 28, wherein the essential oil comprises Ylang-ylang oil.

30. The hair treatment composition according to any one of claims 20 to 29, wherein the vegetable oils comprises Argan Oil and the essential oil comprises Ylang Ylang Oil, Orange Oil or a combination.

31. The hair treatment composition according to any one of claims 20 to 30, wherein the vegetable oil is present in the composition in an amount from about 0.1% to about 10% by weight of the composition; or in an amount from about 1% to about 5% by weight of the composition; or from about 1% to about 4% by weight of the composition.

32. The hair treatment composition according to any one of claims 20 to 31, wherein one or more essential oils are present in the composition in an amount from about 0.1% to about 5% by weight of the composition; or an amount from about 0.2% to about 4% by weight of the composition; or in an amount from about 0.2% to about 3% by weight of the composition; or in an amount from about 1% to about 3% by weight of the composition.

33. The hair treatment composition according to any one of claims 20 to 32, wherein the hair treatment composition is a hair spray.

34. The hair treatment composition according to any one of claims 20 to 32, wherein the hair treatment composition is a gel or lotion.

35. A hair treatment kit comprising:
(i) a hair masque;
(ii) an oil serum comprising:
one or more oils comprising triglycerides of C16-C18 fatty acids, which fatty acids may be saturated or unsaturated; and
(iii) a hyaluronic acid serum comprising:
hyaluronic acid,
optionally, one or more oils comprising triglycerides of C16-C18 fatty acids,
which fatty acids may be saturated or unsaturated,
optionally, a preservative, and
water.

36. The hair treatment kit of claim 35, wherein the masque comprises one or more of 1) one or more C16-C18 fatty alcohols, 2) one or more oils comprising triglycerides of C16-C18 fatty acids, which fatty acids may be saturated or unsaturated, 3) one or more long-chain quaternary ammonium surfactants, 4) dimethicone, 5) isopropyl alcohol, 6) tocopherol, 7) preservative, 8) pH adjusting agent such as citric acid, 9) glycerin (or glycerol), and 10) water.

37. The hair treatment kit of claim 36, wherein the one or more C16-C18 fatty alcohols of the masque comprise cetearyl alcohol and cetyl alcohol.

38. The hair treatment kit of claim 37, wherein the one or more C16-C18 fatty alcohols of the masque are present in an amount from about 1% to about 10% by weight of the masque.

39. The hair treatment kit of claim 36, wherein each of the one or more oils is selected from a vegetable oil, an essential oil, or combinations thereof.

40. The hair treatment kit of claim 39, wherein each of the one or more vegetable oils is selected from Argania Spinosa Kernel Oil (Argan Oil), Açaí palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil and mixtures thereof.

41. The hair treatment kit of claim 39 or 40, wherein the one or more essential oils is selected from Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and mixtures thereof.

42. The hair treatment kit according to any one of claims 35 to 41, wherein the one or more oils of the masque are present in an amount from about 1% to about 15% by weight of the masque.

43. The hair treatment kit according to any one of claims 35 to 42, wherein the one or more oils of the masque are present in an amount from about 1% to about 10% by weight of the masque.

44. The hair treatment kit according to any one of claims 35 to 43, wherein the one or more long-chain quaternary ammonium surfactants of the masque comprise cetrimonium chloride and behentrimonium chloride.

45. The hair treatment kit according to any one of claims 35 to 44, wherein the long-chain quaternary ammonium surfactants of the masque are present in an amount from about 0.1% to about 10% by weight of the masque.

46. The hair treatment kit according to any one of claims 35 to 45, wherein the long-chain quaternary ammonium surfactants of the masque are present in an amount from about 2% to about 5% by weight of the masque.

47. The hair treatment kit according to any one of claims 35 to 46, wherein the preservative of the masque is present in an amount from about 0.1% to about 5% by weight of the masque.

48. The hair treatment kit according to any one of claims 35 to 47, wherein the preservative of the masque is present in an amount up to about 2% by weight of the masque.

49. The hair treatment kit according to any one of claims 35 to 48, wherein the water of the masque is present in an amount not less than about 60% by weight of the masque.

50. The hair treatment kit according to any one of claims 35 to 49, wherein the water the masque is present in an amount not less than about 80% by weight of the masque.

51. The hair treatment kit according to any one of claims 35 to 50, wherein the one or more oils of the oil serum comprises a vegetable oil and optionally an essential oil.

52. The hair treatment kit of claim 51, wherein the one or more vegetable oils comprise one or more of Argania Spinosa Kernel Oil (Argan Oil), Açaí palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil, and mixtures thereof.

53. The hair treatment kit of claim 51 or 52, wherein the one or more essential oils are comprise one or more of Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and mixtures thereof.

54. The hair treatment kit according to any one of claims 35 to 53, wherein the one or more oils of the oil serum are present in an amount from about 50% to about 100% by weight of the oil serum.

55. The hair treatment kit according to any one of claims 35 to 54, wherein the hyaluronic acid of the hyaluronic acid serum has a molecular weight of about 8 kilodaltons or less, or about 5 kilodaltons or less.

56. The hair treatment kit according to any one of claims 35 to 55, wherein the hyaluronic acid of the hyaluronic acid serum has a molecular weight of about 5 kilodaltons or less.

57. The hair treatment kit according to any one of claims 35 to 56, wherein the hyaluronic acid of the hyaluronic acid serum is present in an amount from about 1% to about 20% by weight of the hyaluronic acid serum.

58. The hair treatment kit according to any one of claims 35 to 57, wherein the hyaluronic acid of the hyaluronic acid serum is present in an amount from about 5% to about 15% by weight of the hyaluronic acid serum.

60. The hair treatment kit of claim 36, wherein the one or more oils of the hyaluronic acid serum are selected from Argania Spinosa Kernel Oil (Argan Oil), Açaí palm oil, African Galanga Oil, Almond Oil, Ama Oil, Apricot Seed Oil, Asian Borage Oil, Avocado Oil, Brazil nut oil, Buriti oil, Coconut Oil, Camellia Oil, Castor Oil, Flaxseed Oil (Linseed Oil), Grapeseed Oil, Jojoba Oil, Marula Oil, Olive Oil, Rice bran oil, Shea Oil, Shea Nut Oil, Tea Tree Oil, Agar oil, balsam of Peru, Basil oil, Bay leaf oil, Bergamot oil, Benzoin oil, Birch oil, Calamodin oil, Calamus oil, Cannabis flower oil, Cardamom seed oil, Cedar oil (or cedarwood oil), Chamomile oil, Cinnamon oil, Citron oil, Clary Sage oil, Clove oil, Coriander oil, Cranberry seed oil, Cypress oil, Cypriol oil, Davana oil, Eucalyptus oil, Elemi oil, Fenne oil, Frankincense oil, Gardenia oil, Geranium oil, Ginger root oil, Grapefruit oil, Immortelle oil, Jasmine oil, Juniper oil, Laurel oil, Lavender oil, Lemon oil, Lemongrass oil, Lime oil, Litsea cubeba oil, Lotus oil, Melissa oil, Moringa oil, Mountain Savory, Myrrh, Neem oil or neem tree oil, Neroli oil, Orange oil, Osmanthus oil, d-Limonene, Orris oil, Palmarosa oil, Patchouli oil, Peppermint oil, Rose oil, Rosemary oil, Rosewood oil, Sandalwood oil, Star anise oil, Spearmint oil, Tangerine oil, Tea tree oil, Valerian oil, Vanila oil, Verbena oil, Warionia oil, White Orchid oil, Ylang-ylang oil, and mixtures thereof.

61. The hair treatment kit of claim 60, wherein the one or more oils of the hyaluronic acid serum comprises one or more of Argan Oil, Shea Nut Oil, Vitamin E oil, Avocado Oil, Orange Oil and Ylang Ylang Oil.

62. The hair treatment kit of claim 60, wherein the one or more oils of the hyaluronic acid serum comprises one or more of Argan Oil, Orange oil and Ylang Ylang Oil.

63. The hair treatment kit according to any one of claims 35 to 62, wherein the one or more oils of the hyaluronic acid serum are present in an amount from about 0% to about 10% by weight of the hyaluronic acid serum.

64. The hair treatment kit according to any one of claims 35 to 63, wherein the one or more oils of the hyaluronic acid serum are present in an amount from about 0% to about 5% by weight of the hyaluronic acid serum.

65. The hair treatment kit according to any one of claims 35 to 64, wherein the preservative of the hyaluronic acid serum comprises a preservative selected from DMDM hydantoin, imadozolidinyl urea, diazolidinyl urea, phenoxy ethanol, caprylyl glycol, methylisothiazolinone, benzoic acid, sorbic acid, levulinic acid, anisic acid, methylparaben, ethylparaben, propylparaben, butylparaben, isobutylparaben, benzyl alcohol, piroctone olamine, or combinations thereof.

66. The hair treatment kit of claim 65, wherein the preservative of the hyaluronic acid serum comprises benzyl alcohol.

67. The hair treatment kit according to any one of claims 35 to 66, wherein the preservative of the hyaluronic acid serum is present in an amount from about 0.1% to about 5% by weight of the hyaluronic acid serum.

68. The hair treatment kit according to any one of claims 35 to 67, wherein the preservative of the hyaluronic acid serum is present in an amount up to about 3% by weight, or up to about 2% by weight, of the hyaluronic acid serum.

69. A method for treating a keratinous fiber or a scalp of a subject, the method comprising: applying the hair treatment compositions of any one of claims 1 to 34 to the hair and/or the scalp.

70. The method of claim 69, further comprising a step of washing or rinsing the hair or the scalp prior to applying the hair treatment composition.

71. The method of claim 69 or claim 70, further comprising a step of steaming the treatment into the hair using a flat iron that produces steam.

72. A method for treating the hair of a subject, the method comprising: mixing the masque, oil serum and hyaluronic acid serum of any one of claims 35 to 68, and applying the resultant mixture to the hair.

73. The method of claim 72, further comprising a step of washing or rinsing the hair a prior to applying the hair treatment.

74. The method of claim 72 or 73, further comprising a step of steaming the treatment into the hair using a flat iron that produces steam.

75. The method according to claim 74, further comprising the step of rinsing the treatment from the hair after it is steamed into the hair, followed by optionally allowing the hair to dry naturally or blow drying the hair to the desired style.
